(19) United States
(12) Patent Application Publication
(10) Pub. No.: US 2026/0265653 A1
(43) Pub. Date: Sep. 10, 2026

(54) MULTIFUNCTIONAL LARGE-SCALE CELL CULTURE CHAMBER

(71) Applicant: CellFabs Inc., Tainan City (TW)

(72) Inventors: Yun-Shu Chiou, Tainan City (TW); Shang-Chih Lin, Tainan City (TW)

(73) Assignee: CellFabs Inc., Tainan City (TW)

(21) Appl. No.: 19/071,254

(22) Filed: Mar. 5, 2025

(51) Int. Cl. C12M 1/24; C12M 1/00; C12M 1/12; C12M 1/34; C12M 3/06

(52) U.S. Cl. CPC C12M 23/08; C12M 23/34; C12M 23/38; C12M 23/50; C12M 23/52; C12M 27/16; C12M 37/00; C12M 41/12; C12M 41/26

(57) ABSTRACT

A multifunctional large-scale cell cultivation chamber, wherein a cultivation chamber is internally divided into a cell cultivation area and a maintenance area. Two racks are erected in the cell cultivation area, and a plurality of shaking devices are installed at the racks. The shaking devices are used for placing a cell culture bottle. A transfer equipment is fixed to the cultivation chamber via two tracks, the two tracks extending between the cell cultivation area and the maintenance area. Both ends of a main shaft of the transfer equipment are slidably mounted on the two tracks. Both a first gripper and a second gripper of the transfer equipment are slidably mounted on the main shaft. The first gripper is configured to grip the cell culture bottle, and the second gripper is configured to grip the shaking device. The transfer equipment and the shaking devices can be moved to the maintenance area for servicing, thereby preventing operators from entering the cell cultivation area, ensuring safety, and reducing the risk of contamination.

### 1. Field of the Disclosure

The present invention relates to a large-scale cell cultivation chamber, and more particularly to a multifunctional large-scale cell cultivation chamber that achieves cell cultivation through effective automatic control technology and a safe maintenance mechanism.

### 2. Description of the Prior Art

Conventional cell culture methods primarily utilize small incubators to cultivate a single or a small number of cell culture bottles. While this approach effectively controls environmental parameters such as temperature, humidity, and gas concentration in small-scale operations, it becomes inefficient for large-scale cell production. When a large quantity of cells needs to be cultured, multiple incubators must be added, or multiple culture operations must be performed, which not only increases equipment costs but also complicates operations. Additionally, frequent opening and closing of small incubators for placing and retrieving culture bottles, performing media exchanges, or conducting inspections may expose the cell culture environment to external influences, such as temperature fluctuations or contamination risks. Consequently, conventional small incubator methods struggle to meet the demands of modern biomedical and industrial-scale cell cultivation.

To overcome the limitations of small incubators, the industry has introduced the technology of large-scale cell cultivation chambers. Compared to conventional incubators, large-scale cell cultivation chambers offer greater capacity, allowing for the accommodation of multi-layered cell culture racks and a large number of cell culture bottles. However, while this technology enhances the scale of cell cultivation, it also presents significant challenges. Firstly, due to the vast number of cell culture bottles, efficiently arranging, retrieving, and monitoring them becomes a major challenge. In particular, processes such as media exchange, inspection, and sample collection still rely on manual operations, which not only increase the workload of operators but also heighten the risk of errors and contamination.

Moreover, conventional large-scale cell cultivation chambers typically require operators to enter the environment to perform cultivation tasks, such as changing culture medium, relocating culture bottles, or conducting monitoring. However, these operations expose operators to high humidity, elevated temperatures, and high gas concentrations for extended periods, which not only affect operational comfort but also increase physiological stress and pose potential health risks. Additionally, since the cell cultivation area must generally be maintained under sterile conditions, each instance of personnel entry may introduce external contaminants such as dust, bacteria, or viruses, leading to contamination of the cultivation environment and compromising the stability of cell culture. Therefore, reducing the risk of contamination from personnel entering the cell cultivation area while enhancing operational safety and convenience remains a significant challenge in existing technologies.

Conventional large-scale cell cultivation chambers typically require operators to enter the environment to perform cultivation tasks, such as changing culture medium, relocating culture bottles, or conducting monitoring. However, these operations expose operators to high humidity, elevated temperatures, and high gas concentrations for extended periods, which not only affect operational comfort but also increase physiological stress and pose potential health risks. Additionally, since the cell cultivation area must generally be maintained under sterile conditions, each instance of personnel entry may introduce external contaminants such as dust, bacteria, or viruses, leading to contamination of the cultivation environment and compromising the stability of cell culture. Therefore, reducing the risk of contamination from personnel entering the cell cultivation area while enhancing operational safety and convenience remains a significant challenge in existing technologies.

In view of the above, based on years of experience in the manufacturing, development, and design of related products, the inventor has conducted detailed design and careful evaluation to ultimately achieve the present invention, which possesses practical utility.

### SUMMARY OF THE DISCLOSURE

The technical problem that the present invention aims to solve is to address the aforementioned deficiencies in existing technology by providing a multifunctional large-scale cell cultivation chamber.

A cultivation chamber is structured in the shape of a square cabinet enclosed by a front wall, a rear wall, two side walls, a roof panel, and a floor. The interior of the cultivation chamber is divided by a door panel into a cell cultivation area and a maintenance area, wherein the cell cultivation area is adjacent to the front wall, and the maintenance area is adjacent to the rear wall. Two racks are erected parallel to the side walls within the cell cultivation area, and multiple bases are arranged in a matrix configuration at the racks. A culture bottle window is provided on the front wall. A plurality of shaking devices are installed at the bases, each shaking device being provided with a shaking plate, the shaking plate being configured to hold a cell culture bottle. A transfer equipment comprises two tracks, a main shaft, a first gripper, and a second gripper, wherein the two tracks are respectively fixed to the roof panel and the floor, and both tracks are parallel to the racks. One end of the tracks is adjacent to the front wall, while the other end extends into the maintenance area. One end of the main shaft is connected to a steering mechanism, while the other end is connected to a suspension member. The steering mechanism is slidably mounted on the track at the floor, whereas the suspension member is slidably mounted on the track at the roof panel. The first gripper and the second gripper are both slidably mounted on the main shaft. The first gripper is configured to grip the cell culture bottle and transport it between the culture bottle window and the shaking plate. The second gripper is configured to grip the shaking device and transport it between the maintenance area and the base.

The shaking device comprises a plate body, an actuator, at least one lifting mechanism, and a limiting frame, wherein the actuator and the lifting mechanism are both fixed to the plate body. The actuator is connected to a tilted seat, and the lifting mechanism is connected to an inclined block. The center of the shaking plate is connected to an extension member, and an insert rod extends through the extension member and the tilted seat. The limiting frame is disposed between the shaking plate and the plate body and is in contact with the inclined block. When the limiting frame descends, the shaking plate is inclined, and when the limiting frame is raised by the lifting mechanism, it supports the shaking plate in a horizontal position.

The base of the rack is formed with two sliding slots and two ball slots. The plate body of the shaking device is provided with two spherical bodies, and when the plate body is slidably positioned in the sliding slots, the two spherical bodies are restricted within the two ball slots. The plate body is further provided with a clamping member, and the second gripper clamps the clamping member to extract the shaking device by counteracting the resistance between the spherical bodies and the ball slots.

The bottom of the cell culture bottle is recessed with a plurality of positioning recesses, and the shaking plate is correspondingly provided with a plurality of positioning projections, wherein the engagement between the positioning recesses and the positioning projections secures the cell culture bottle in place. The limiting frame is provided with an infrared sensor, which is configured to detect whether the cell culture bottle is placed on the shaking plate.

The cell culture bottle is sealed with a cap, and a sensor host is fixed to the exterior of the cap. The sensor host is electrically connected to a sensor rod, which is inserted into the interior of the cell culture bottle through the cap. The sensor rod is used to measure the temperature and pH value of the culture medium inside the bottle.

The cultivation chamber is provided with a flow guide area between each of the two side walls and the two racks. A temperature control device is installed at the interface between the racks and the flow guide areas. The temperature control device comprises a plurality of fans, a plurality of thermistors, and a plurality of thermistor strips. The fans and thermistors are installed in groups on the racks and are positioned near the roof panel and the floor. The thermistor strips are installed in the flow guide areas. The fans blow air toward the flow guide areas, directing the heated air generated by the thermistors and the thermistor strips toward the cell culture bottles.

The steering mechanism comprises a frame, a steering motor, a tubular body, a load-bearing seat, a lower gear, and an upper gear. The steering mechanism is slidably mounted on the track via the frame. The steering motor is fixed to the frame and is provided with a steering column protruding upward. The tubular body is connected to the frame, and the steering column is positioned inside the tubular body and connected to the lower gear. The load-bearing seat is connected to the tubular body and is internally equipped with a tapered roller bearing and a thrust bearing. The main shaft coaxially extends with a load-bearing rod and a steering rod. The load-bearing rod extends through the load-bearing seat and is supported by the tapered roller bearing and the thrust bearing, while the steering rod is positioned within the tubular body and connected to the upper gear. The lower gear and the upper gear are engaged with each other, allowing the steering motor to operate without bearing the weight of the main shaft.

The first gripper is provided with a first motor, which drives the first gripper to perform a gripping operation. The first gripper is connected to a slider and is laterally slidable on a lifting arm via the slider. The lifting arm is longitudinally slidable on the main shaft and is equipped with a third motor, which moves the slider.

The second gripper is provided with a second motor, which drives the second gripper to perform a gripping operation. The second gripper is connected to a displacement block and is laterally slidable on a horizontal arm via the displacement block. The horizontal arm is longitudinally slidable on the main shaft and is equipped with a fourth motor, which moves the displacement block.

A storage platform extends outward from the culture bottle window, and a UV lamp is positioned directly above the storage platform. After the first gripper places the cell culture bottle on the storage platform, or before the first gripper grips the cell culture bottle from the storage platform, the UV lamp is used to sterilize the surface of the cap of the cell culture bottle.

The main objective of the present invention is to divide the interior of the cultivation chamber into a cell cultivation area and a maintenance area using a door panel, wherein the cell cultivation area is used to accommodate multiple cell culture bottles. One end of the track is adjacent to the front wall, while the other end extends into the maintenance area, allowing the main shaft to move along the two tracks. Both the first gripper and the second gripper are slidably mounted on the main shaft, enabling vertical movement. The first gripper is configured to grip the cell culture bottle and transport it between the culture bottle window and the shaking plate, while the second gripper is configured to grip the shaking device and transport it between the maintenance area and the base. When the transfer equipment or the shaking device requires maintenance or servicing, the transfer equipment can be moved to the maintenance area, eliminating the need for operators to enter the cell cultivation area. This design prevents operators from being exposed to the high carbon dioxide concentration within the cell cultivation area, thereby significantly improving operator safety while also reducing the risk of contamination in the cell cultivation area.

Other objectives, advantages, and novel features of the present invention will become more apparent from the following detailed description and the accompanying drawings.

### BRIEF DESCRIPTION OF THE DRAWINGS

The present invention will be best understood by referring to the following detailed description of one illustrative embodiment in conjunction with the accompanying drawings, in which

FIG. 1 is a perspective view of the present invention;

FIG. 2 is a top-view schematic diagram of the cultivation chamber of the present invention;

FIG. 3 is a longitudinal sectional perspective view of the present invention;

FIG. 4 is a transverse sectional perspective view of the present invention;

FIG. 5 is a perspective view of the transfer equipment of the present invention;

FIG. 6 is a perspective view of the present invention showing the first gripper holding a cell culture bottle;

FIG. 7 is a partial schematic diagram of the transfer equipment of the present invention;

FIG. 8 is a partial sectional view of the transfer equipment of the present invention;

FIG. 9 is a perspective view of the transfer equipment main shaft positioned in the cell cultivation area of the present invention;

FIG. 10 is a perspective view of the transfer equipment main shaft positioned in the maintenance area of the present invention;

FIG. 11 is a top-view schematic diagram of the cultivation chamber of the present invention;

FIG. 12 is an exploded perspective view of the shaking device of the present invention;

FIG. 13 is a perspective view of the shaking device of the present invention;
and

FIG. 14 is an operational schematic diagram of the present invention showing the process of retrieving the shaking device.

### DETAILED DESCRIPTION

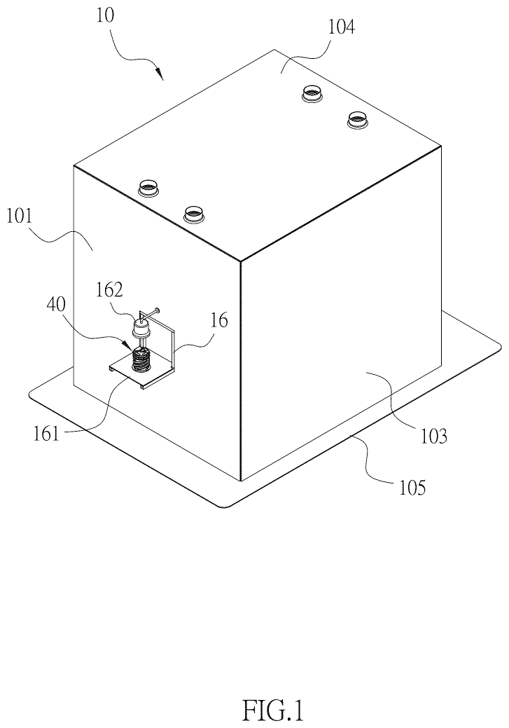

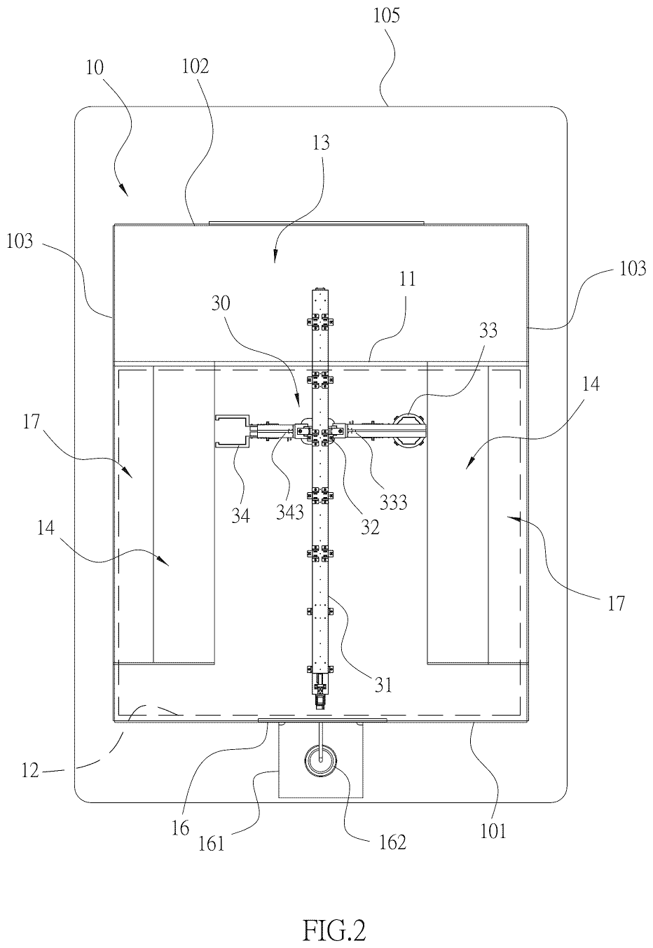

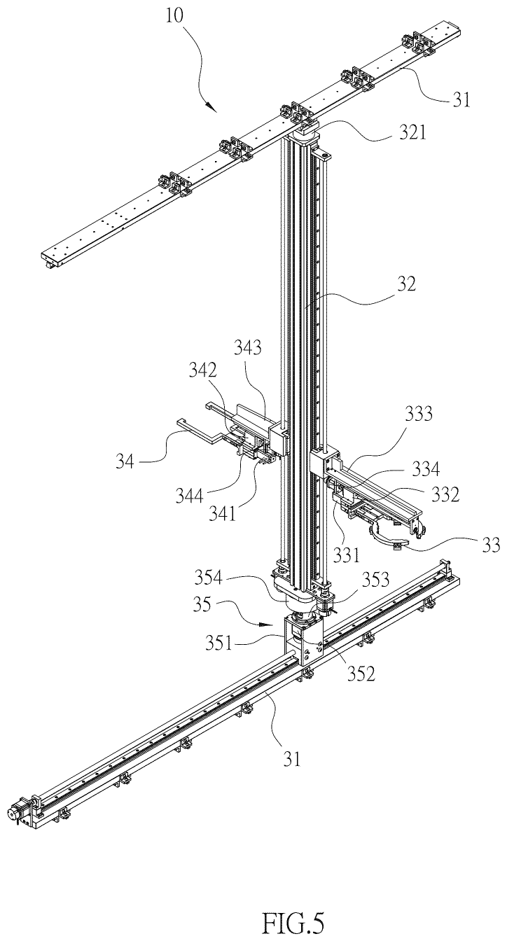

Referring to FIGS. 1 through 5, a multifunctional large-scale cell cultivation chamber comprises a cultivation chamber 10, a plurality of shaking devices 20, and a transfer equipment 30. The cultivation chamber 10 is structured in the shape of a square cabinet enclosed by a front wall 101, a rear wall 102, two side walls 103, a roof panel 104, and a floor 105. The interior of the cultivation chamber 10 is divided by a door panel 11 into a cell cultivation area 12 and a maintenance area 13, wherein the cell cultivation area 12 is adjacent to the front wall 101, and the maintenance area 13 is adjacent to the rear wall 102. Two racks 14 are erected parallel to the side walls 103 within the cell cultivation area 12, and multiple bases 15 are arranged in a matrix configuration at the racks 14. A culture bottle window 16 is provided on the front wall 101.A plurality of shaking devices 20 are installed at the bases 15, each shaking device 20 being provided with a shaking plate 21, which is configured to hold a cell culture bottle 40. The cell culture bottle 40 is sealed with a cap 41, and a sensor host 42 is fixed to the exterior of the cap 41. The sensor host 42 is electrically connected to a sensor rod 43, which is inserted into the interior of the cell culture bottle 40 through the cap 41. The sensor rod 43 is used to measure the temperature and pH value of the culture medium inside the bottle 40, thereby enabling automated cell cultivation through data analysis. The transfer equipment 30 comprises two tracks 31, a main shaft 32, a first gripper 33, a second gripper 34, and a steering mechanism 35. The two tracks 31 are respectively fixed to the roof panel 104 and the floor 105, and both tracks 31 are parallel to the racks 14. One end of each track 31 is adjacent to the front wall 101, while the other end extends into the maintenance area 13. One end of the main shaft 32 is connected to the steering mechanism 35, while the other end is connected to a suspension member 321. The steering mechanism 35 is slidably mounted on the track 31 at the floor 105, and the suspension member 321 is slidably mounted on the track 31 at the roof panel 104, allowing the main shaft 32 to move along the two tracks 31. The first gripper 33 and the second gripper 34 are both slidably mounted on the main shaft 32, enabling vertical movement. The first gripper 33 is configured to grip the cell culture bottle 40 and transport it between the culture bottle window 16 and the shaking plate 21. The second gripper 34 is configured to grip the shaking device 20 and transport it between the maintenance area 13 and the base 15.

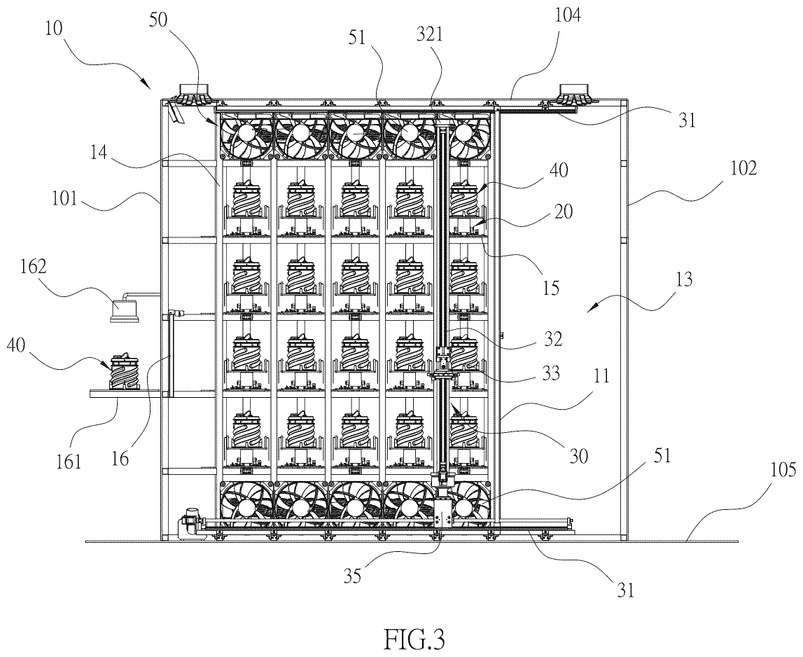

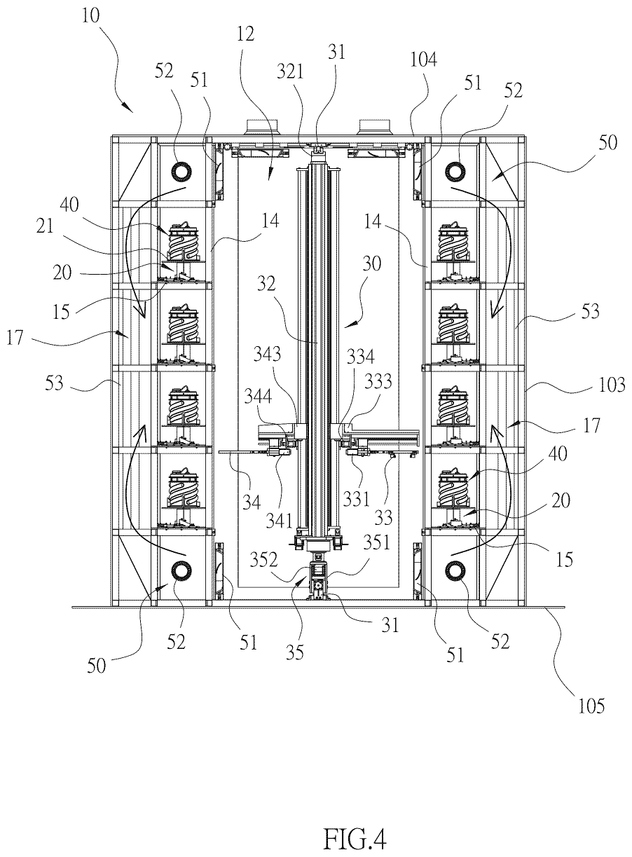

Referring to FIGS. 3 and 4, a flow guide area 17 is provided between each of the two side walls 103 and the two racks 14 of the cultivation chamber 10. A temperature control device 50 is installed at the interface between the racks 14 and the flow guide areas 17. The temperature control device 50 comprises a plurality of fans 51, a plurality of thermistors 52, and a plurality of thermistor strips 53. The fans 51 and thermistors 52 are installed in groups on the racks 14 and are positioned near the roof panel 104 and the floor 105, while the thermistor strips 53 are installed in the flow guide areas 17. The fans 51 blow air toward the flow guide areas 17, directing the heated air generated by the thermistors 52 and the thermistor strips 53 toward the cell culture bottles 40. In combination with the sensor rod 43, which measures the temperature of the culture medium inside the bottles, this system enables the adjustment of the cultivation temperature for multiple cell culture bottles 40 within the cultivation chamber 10. This design is particularly suitable for large-scale cell cultivation chambers, ensuring that each cell culture bottle 40 maintains an optimal temperature for cell growth.

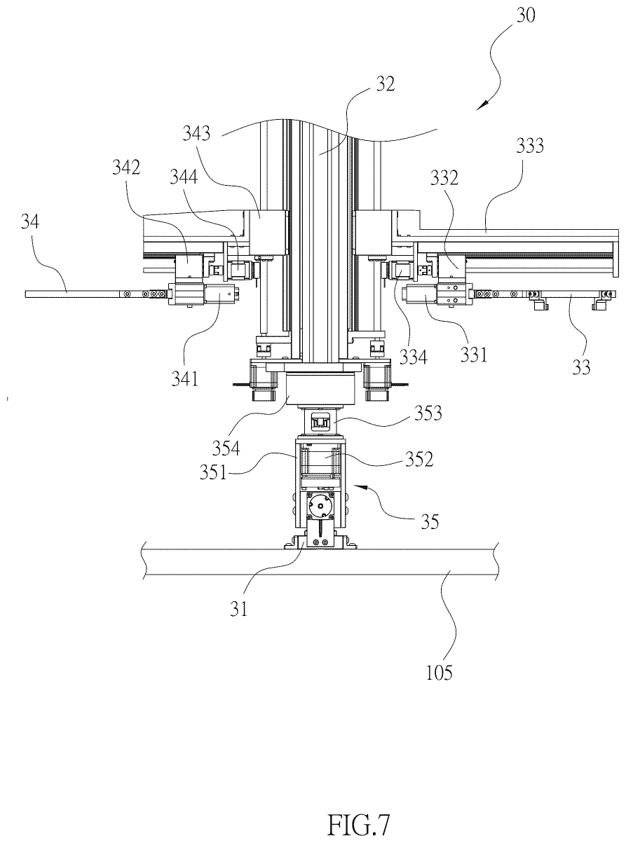

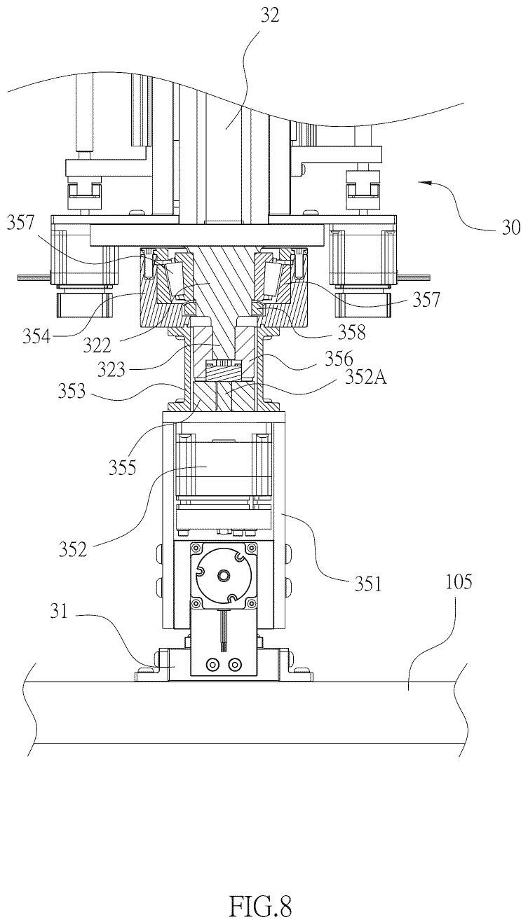

Referring to FIGS. 7 and 8, the steering mechanism 35 comprises a frame 351, a steering motor 352, a tubular body 353, a load-bearing seat 354, a lower gear 355, and an upper gear 356. The steering mechanism 35 is slidably mounted on the track 31 via the frame 351. The steering motor 352 is fixed to the frame 351 and is provided with a steering column 352A protruding upward. The tubular body 353 is connected to the frame 351, and the steering column 352A is positioned inside the tubular body 353 and connected to the lower gear 355. The load-bearing seat 354 is connected to the tubular body 353 and is internally equipped with a tapered roller bearing 357 and a thrust bearing 358. The main shaft 32 coaxially extends with a load-bearing rod 322 and a steering rod 323. The load-bearing rod 322 extends through the load-bearing seat 354 and is supported by the tapered roller bearing 357 and the thrust bearing 358, allowing the weight of the main shaft 32, the first gripper 33, and the second gripper 34 to be transmitted to the load-bearing seat 354. The integrated assembly of the load-bearing seat 354, the tubular body 353, and the frame 351 supports the total weight. The steering rod 323 is positioned inside the tubular body 353 and connected to the upper gear 356, while the lower gear 355 and the upper gear 356 are engaged with each other. The upper gear 356 exerts downward pressure on the lower gear 355 under load, ensuring that the steering motor 352 does not bear the weight of the main shaft 32. This design enables the steering motor 352 to drive the main shaft 32 without load, significantly improving the durability of the steering motor 352.

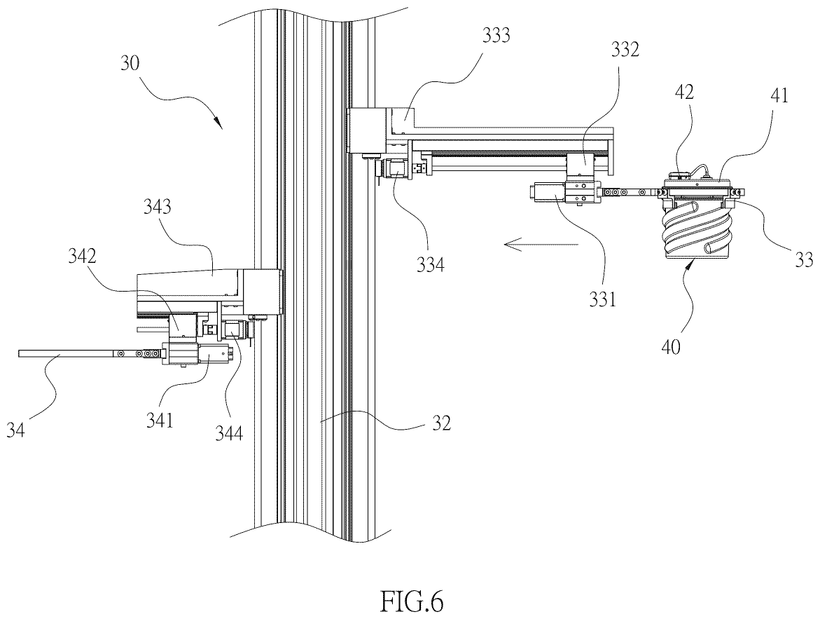

Referring to FIGS. 6, 7, and 8, the first gripper 33 is provided with a first motor 331, which drives the first gripper 33 to perform a gripping operation. The first gripper 33 is connected to a slider 332, and the first gripper 33 is slidably mounted on a lifting arm 333 via the slider 332. The lifting arm 333 is longitudinally slidable along the main shaft 32 and is equipped with a third motor 334, which moves the slider 332, allowing the first gripper 33 to achieve horizontal displacement along the lifting arm 333. The lifting arm 333 is used to control the height position of the first gripper 33, enabling it to grip cell culture bottles 40 at different positions and transport them along the track 31 to the front wall 101. The steering mechanism 35 then rotates the main shaft 32, allowing the first gripper 33 to pass through the culture bottle window 16, thereby achieving the practical function of automatically retrieving or placing the cell culture bottle 40 into or from the cultivation chamber 10. Furthermore, a storage platform 161 extends outward from the culture bottle window 16, and a UV lamp 162 is positioned directly above the storage platform 161. After the first gripper 33 places the cell culture bottle 40 on the storage platform 161, or before the first gripper 33 grips the cell culture bottle 40 from the storage platform 161, the UV lamp 162 is used to sterilize the surface of the cap 41 of the cell culture bottle 40.

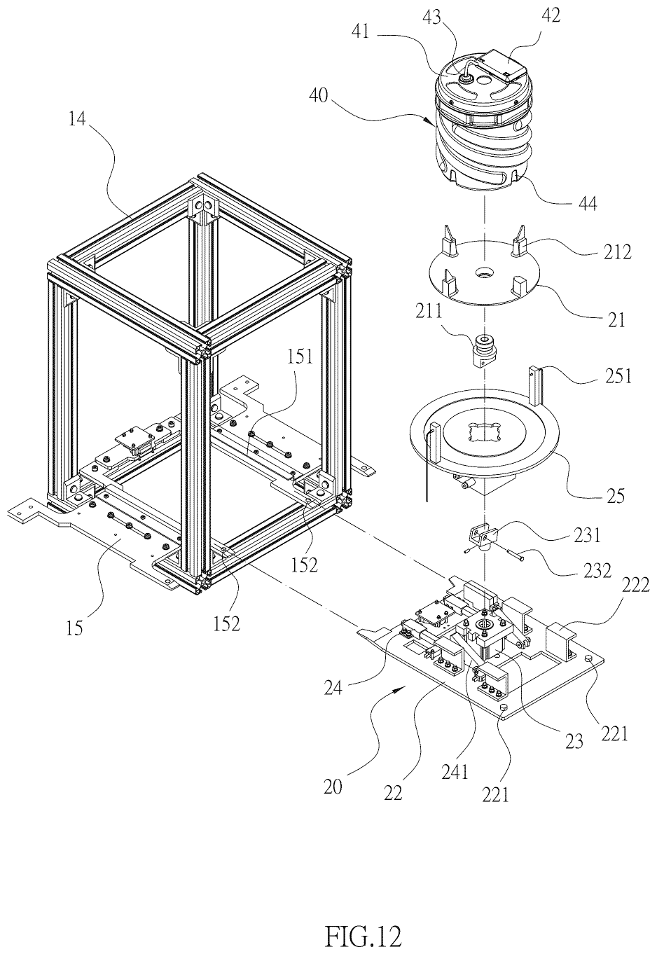

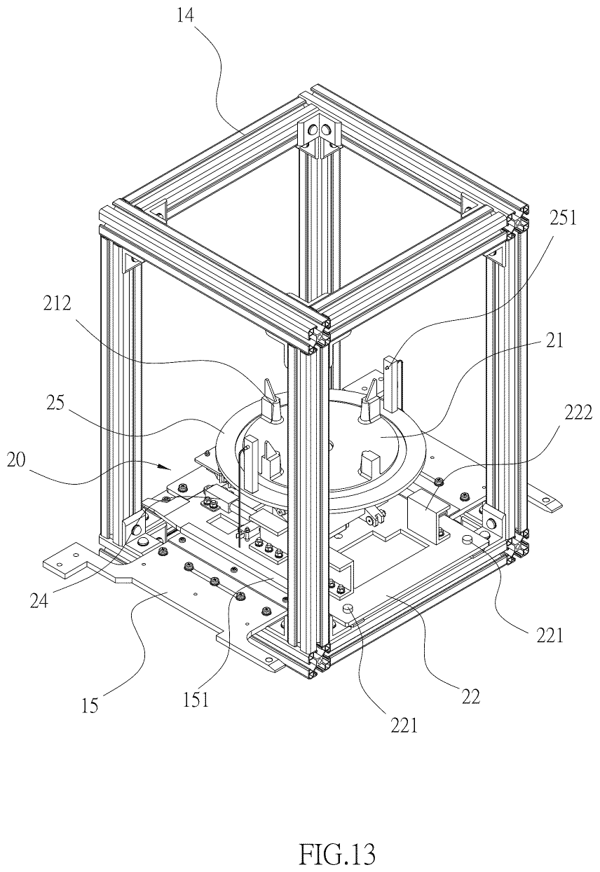

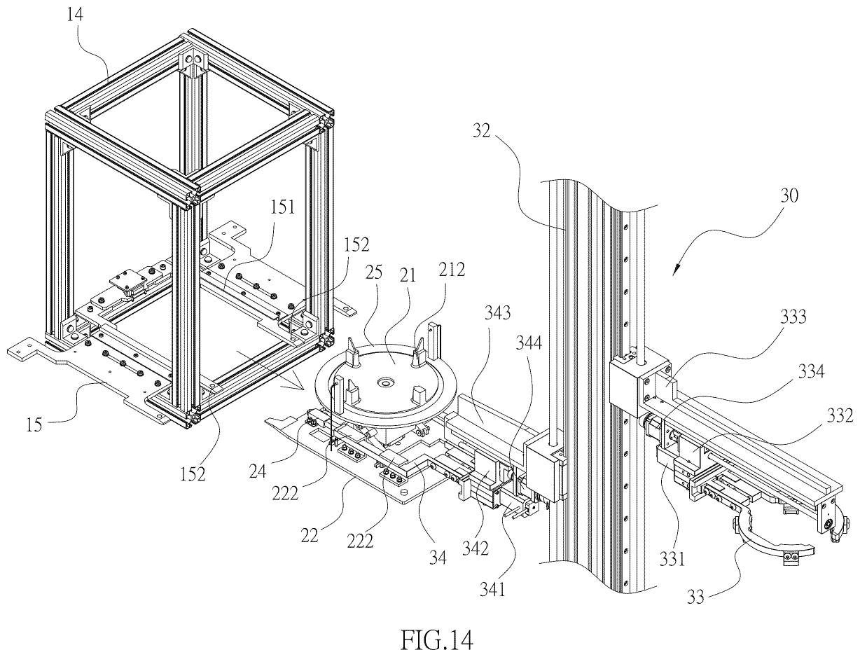

Referring to FIGS. 12 through 14, the shaking device 20 further comprises a plate body 22, an actuator 23, at least one lifting mechanism 24, and a limiting frame 25. The actuator 23 and the lifting mechanism 24 are both fixed to the plate body 22. The actuator 23 is connected to a tilted seat 231, and the lifting mechanism 24 is connected to an inclined block 241. The center of the shaking plate 21 is connected to an extension member 211, and an insert rod 232 extends through the extension member 211 and the tilted seat 231, allowing the extension member 211 to oscillate around the insert rod 232 as a pivot point. The limiting frame 25 is disposed between the shaking plate 21 and the plate body 22 and is in contact with the inclined block 241. When the limiting frame 25 descends, the shaking plate 21 tilts. When the limiting frame 25 is raised by the inclined block 241 of the lifting mechanism 24, it supports the shaking plate 21 in a horizontal position, allowing the first gripper 33 to smoothly retrieve the cell culture bottle 40. When the limiting frame 25 is raised, it supports the shaking plate 21 in a horizontal position. At this point, the extension member 211 is lifted and oscillates around the insert rod 232, keeping the shaking plate 21 level. When the limiting frame 25 descends, the extension member 211 swings downward and is restricted by the tilted seat 231, causing the shaking plate 21 to tilt. The actuator 23, through the tilted seat 231, drives the extension member 211, allowing the shaking plate 21 to perform a three-dimensional circular shaking motion, thereby effectively enhancing the shaking effect. The bottom of the cell culture bottle 40 is recessed with a plurality of positioning recesses 44, and the shaking plate 21 is correspondingly provided with a plurality of positioning projections 212, securing the cell culture bottle 40 in place through the engagement of the positioning recesses 44 and the positioning projections 212. Additionally, the limiting frame 25 is provided with an infrared sensor 251, which detects whether the cell culture bottle 40 is placed on the shaking plate 21, assisting in achieving automated control.

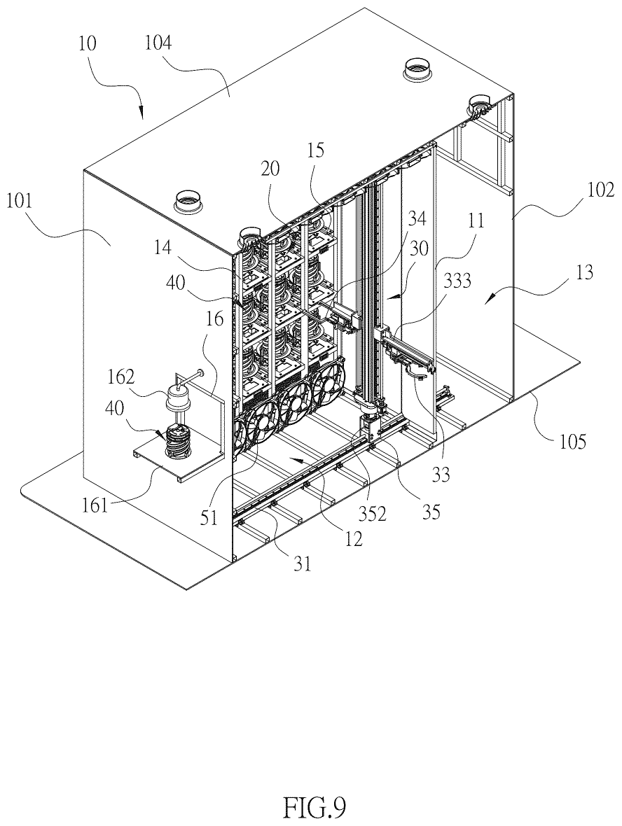

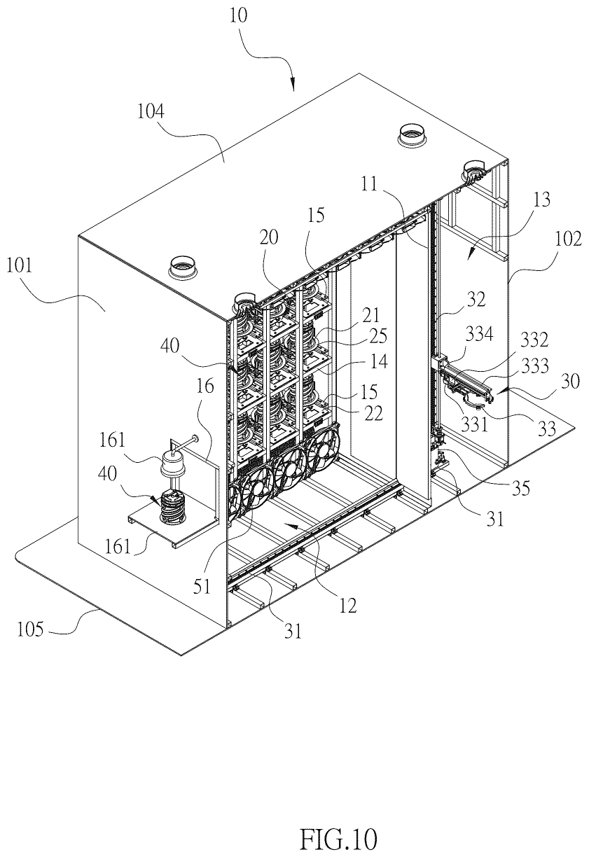

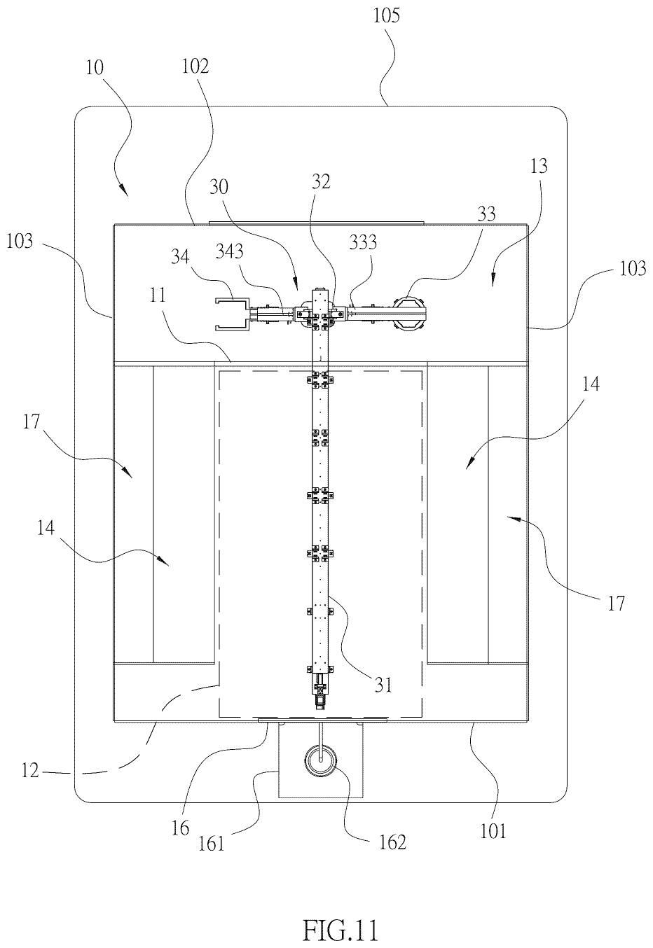

Referring to FIGS. 9 through 11, the second gripper 34 is used to grip the shaking device 20 and transport it between the maintenance area 13 and the base 15. The second gripper 34 is equipped with a second motor 341, which drives the second gripper 34 to perform a gripping operation. The second gripper 34 is connected to a displacement block 342 and is laterally slidable on a horizontal arm 343 via the displacement block 342. The horizontal arm 343 is longitudinally slidable along the main shaft 32 and is equipped with a fourth motor 344, which moves the displacement block 342. Further, the base 15 of the rack 14 is formed with two sliding slots 151 and two ball slots 152. The plate body 22 of the shaking device 20 is provided with two spherical bodies 221, and when the plate body 22 is slidably positioned in the sliding slots 151, the two spherical bodies 221 are restricted within the two ball slots 152. The plate body 22 is further provided with a clamping member 222, which is gripped by the second gripper 34 for extraction. By counteracting the resistance between the spherical bodies 221 and the ball slots 152, the second gripper 34 enables the rapid removal of the shaking device 20. The transfer equipment 30 then transports the shaking device 20 to the maintenance area 13. During this process, the door panel 11 temporarily opens in coordination with the transfer equipment 30, and once the main shaft 32 enters the maintenance area 13, the door panel 11 quickly closes. This allows the shaking device 20 to undergo maintenance and servicing in the maintenance area 13. Similarly, when the transfer equipment 30 itself requires maintenance or servicing, it can be moved to the maintenance area 13. This design eliminates the need for operators to enter the cell cultivation area 12, preventing exposure to the high carbon dioxide concentration inside the cell cultivation area 12. As a result, it effectively enhances operator safety while also reducing the risk of contamination in the cell cultivation area.

The foregoing description is merely a preferred embodiment of the present invention and should not be construed as limiting the scope of its implementation. Any equivalent modifications or variations made in accordance with the scope of the claims of the present invention shall still fall within the scope covered by the present patent.

What is claimed is:

1. A multifunctional large-scale cell cultivation chamber, comprising:
a cultivation chamber, which is structured in the shape of a square cabinet enclosed by a front wall, a rear wall, two side walls, a roof panel, and a floor, wherein the interior of the cultivation chamber is divided by a door panel into a cell cultivation area and a maintenance area, the cell cultivation area being adjacent to the front wall and the maintenance area being adjacent to the rear wall, wherein two racks are erected parallel to the side walls within the cell cultivation area, and multiple bases are arranged in a matrix configuration at the racks, wherein a culture bottle window is provided on the front wall;
a plurality of shaking devices installed at the bases, each shaking device being provided with a shaking plate, the shaking plate being configured to hold a cell culture bottle;
a transfer equipment comprising two tracks, a main shaft, a first gripper, and a second gripper, wherein the two tracks are respectively fixed to the roof panel and the floor, both tracks being parallel to the racks, wherein one end of the tracks is adjacent to the front wall, and the other end extends into the maintenance area, wherein one end of the main shaft is connected to a steering mechanism, and the other end is connected to a suspension member, wherein the steering mechanism is slidably mounted on the track at the floor, and the suspension member is slidably mounted on the track at the roof panel, wherein the first gripper and the second gripper are both slidably mounted on the main shaft, the first gripper being configured to grip the cell culture bottle and transport it between the culture bottle window and the shaking plate, and the second gripper being configured to grip the shaking device and transport it between the maintenance area and the base.

2. The multifunctional large-scale cell cultivation chamber according to claim 1, wherein the shaking device comprises a plate body, an actuator, at least one lifting mechanism, and a limiting frame, wherein the actuator and the lifting mechanism are both fixed to the plate body, the actuator being connected to a tilted seat and the lifting mechanism being connected to an inclined block, wherein a shaking plate is centrally connected to an extension member, and an insert rod extends through the extension member and the tilted seat, wherein the limiting frame is disposed between the shaking plate and the plate body and is in contact with the inclined block, wherein when the limiting frame descends, the shaking plate is inclined, and when the limiting frame is raised by the lifting mechanism, it supports the shaking plate in a horizontal position.

3. The multifunctional large-scale cell cultivation chamber according to claim 2, wherein the base of the rack is formed with two sliding slots and two ball slots, wherein the plate body of the shaking device is provided with two spherical bodies, and when the plate body is slidably positioned in the sliding slots, the two spherical bodies are restricted within the two ball slots, wherein the plate body is further provided with a clamping member, and the second gripper clamps the clamping member to extract the shaking device by counteracting the resistance between the spherical bodies and the ball slots.

4. The multifunctional large-scale cell cultivation chamber according to claim 2, wherein the bottom of the cell culture bottle is recessed with a plurality of positioning recesses, and the shaking plate is correspondingly provided with a plurality of positioning projections, wherein the engagement between the positioning recesses and the positioning projections secures the cell culture bottle in place, wherein the limiting frame is provided with an infrared sensor, the infrared sensor being configured to detect whether the cell culture bottle is placed on the shaking plate.

5. The multifunctional large-scale cell cultivation chamber according to claim 1, wherein the cell culture bottle is sealed with a cap, the cap having a sensor host fixed to its exterior, wherein the sensor host is electrically connected to a sensor rod, and the sensor rod is inserted into the interior of the cell culture bottle through the cap, the sensor rod being configured to measure the temperature and pH value of the culture medium inside the bottle.

6. The multifunctional large-scale cell cultivation chamber according to claim 5, wherein a storage platform extends outward from the culture bottle window, and a UV lamp is positioned above the storage platform, wherein after the first gripper places the cell culture bottle on the storage platform, or before the first gripper grips the cell culture bottle from the storage platform, the UV lamp is used to sterilize the surface of the cap of the cell culture bottle.

7. The multifunctional large-scale cell cultivation chamber according to claim 1, wherein the steering mechanism comprises a frame, a steering motor, a tubular body, a load-bearing seat, a lower gear, and an upper gear, wherein the steering mechanism is slidably mounted on the track via the frame, the steering motor is fixed to the frame and is provided with a steering column protruding upward, wherein the tubular body is connected to the frame, and the steering column is positioned inside the tubular body and connected to the lower gear, wherein the load-bearing seat is connected to the tubular body and is internally equipped with a tapered roller bearing and a thrust bearing, wherein the main shaft coaxially extends with a load-bearing rod and a steering rod, the load-bearing rod extends through the load-bearing seat and is supported by the tapered roller bearing and the thrust bearing, while the steering rod is positioned within the tubular body and connected to the upper gear, wherein the lower gear and the upper gear are engaged with each other, allowing the steering motor to operate without bearing the weight of the main shaft.

8. The multifunctional large-scale cell cultivation chamber according to claim 1, wherein the first gripper is provided with a first motor, the first motor driving the first gripper to perform a gripping operation, wherein the first gripper is connected to a slider, and the first gripper is laterally slidable on a lifting arm via the slider, wherein the lifting arm is longitudinally slidable on the main shaft, and the lifting arm is provided with a third motor, the third motor being configured to move the slider.

9. The multifunctional large-scale cell cultivation chamber according to claim 1, wherein the second gripper is provided with a second motor, the second motor driving the second gripper to perform a gripping operation, wherein the second gripper is connected to a displacement block, and the second gripper is laterally slidable on a horizontal arm via the displacement block, wherein the horizontal arm is longitudinally slidable on the main shaft, and the horizontal arm is provided with a fourth motor, the fourth motor being configured to move the displacement block.

10. The multifunctional large-scale cell cultivation chamber according to claim 1, wherein a flow guide area is provided between each of the two side walls and the two racks, and a temperature control device is disposed at the interface between the racks and the flow guide areas, wherein the temperature control device comprises a plurality of fans, a plurality of thermistors, and a plurality of thermistor strips, wherein the fans and the thermistors are installed in groups on the racks and are positioned near the roof panel and the floor, wherein the thermistor strips are installed in the flow guide areas, wherein the fans blow air toward the flow guide areas, directing the heated air generated by the thermistors and the thermistor strips toward the cell culture bottles.
